(19) United States
(12) Patent Application Publication
(10) Pub. No.: US 2010/0159039 A1
(43) Pub. Date: Jun. 24, 2010

(54) ANTIVIRAL AGENTS CONTAINING A FERMENTATION PRODUCT OF GARLIC BY LACTIC ACID BACTERIA

(75) Inventors: Jong-Hwa LEE, Anseong-shi (KR); Ji-Hyoung WOO, Cheonan-shi (KR); Jung-Sook LEE, Daejeon (KR); Jong-Seog AHN, Daejeon (KR)

Correspondence Address: LEXYOUME IP GROUP, LLC, 5180 PARKSTONE DRIVE, SUITE 175, CHANTILLY, VA, 20151, US

(73) Assignees: Liisna Inc., Cheonyan-city (KR); KOREA RESEARCH INSTITUTE OF BIOSCIENCE AND BIOTECHNOLOGY, Daejeon-city (KR)

(21) Appl. No.: 12/718,679

(22) Filed: Mar. 5, 2010

Related U.S. Application Data
(62) Division of application No. 12/330,641, filed on Dec. 9, 2008.

(51) Int. Cl. A61K 36/8962

(52) U.S. Cl. 424/754

(57) ABSTRACT

An antiviral substance including a fermentation product of garlic that is fermented by lactic acid bacteria is provided. More specifically, an antiviral agent including a fermentation product of garlic, that is fermented by lactic acid bacteria, as an active ingredient is provided. The antiviral agent including a fermentation product of garlic fermented by lactic acid bacteria is useful as a preventing or treating agent of viral infection by inhibiting viruses.

### CROSS-REFERENCE TO RELATED APPLICATION

This application is a divisional of application Ser. No. 12/330,641 filed on Dec. 9, 2008, the entire contents of which are incorporated herein by reference.

### BACKGROUND OF THE INVENTION

(a) Field of the Invention

A novel and efficient substance with antiviral activity, which is capable of replacing conventionally used antiviral agents, is provided. More specifically, an antiviral agent with antiviral activity against pathogenic viruses containing a fermentation product of garlic fermented by lactic acid bacteria as an active ingredient is provided.

(b) Description of the Related Art

Influenza viruses are prevalent in temperate regions during the cold season every year, and cause highly infectious acute respiratory diseases, causing many deaths. Influenza viruses are characterized in that their surface antigens, hemmaglutinin (H) and neuraminidase (N), are modified, causing many antigenic variations. In particular, pandemic influenza virus Type A, which occurs throughout the world every 10-40 years, poses a great threat to human beings. As recognized from the case of the pandemic of avian influenza virus first detected in Hong Kong in 1997, the appearance of new species of influenza viruses can be the beginning of a worldwide disaster. For this reason, the WHO and many countries are preparing measures against influenza pandemics.

Influenza may have no significant effect on healthy people, but it is very serious to old and/or feeble persons or high risk patients with chronic diseases, in some cases causing death. Vaccination against influenza virus has been considered as an effective prevention means. However, recent studies have revealed that the prevention efficiency is approximately 44%, indicating that complete prevention cannot be achieved. Therefore, it is required to develop anti-influenza virus agents that are capable of replacing vaccines.

Neuraminidase inhibitors such as anamivir and oseltamivir, M2 inhibitors such as amantadine and rimantadine, and the like have been used. However, the neuraminidase inhibitors have a disadvantage in that they should be administered within 48 hours after infection. M2 inhibitors are also disadvantageous, since it has been reported that the resistance rate of influenza virus Type B to the inhibitors reaches 100%. In addition, it has been also found that the resistance rate to neuraminidase inhibitors is 10-20%.

Therefore, there is a strong need for urgent development of an antiviral agent having application safety and efficient antiviral activity, without involving resistance problems.

### SUMMARY OF THE INVENTION

Therefore, an object of an embodiment of the present invention is to provide a novel technology to prevent pathogenic viruses.

More specifically, an embodiment of the present invention provides a natural antiviral agent containing one or more selected from the group consisting of a fermentation product of garlic fermented by using lactic acid bacteria, a concentrate of the same, a dried substance of the same, and a combination thereof.

Another embodiment of the present invention provides a composition for preventing and/or treating viral infection, containing a natural antiviral agent as an active ingredient, wherein the natural antiviral agent contains one or more selected from the group consisting of a fermentation product of garlic fermented by using lactic acid bacteria, a concentrate of the same, a dried substance of the same, and a combination thereof.

Another embodiment of the present invention provides a method of preventing and/or treating viral infection by using a natural antiviral agent containing one or more selected from the group consisting of a fermentation product of garlic fermented by using lactic acid bacteria, a concentrate of the same, a dried substance of the same, and a combination thereof.

### DETAILED DESCRIPTION OF THE EMBODIMENTS

Although it is well-known that garlic exerts antiviral and anti-cancer effects, no microorganisms have been found that are capable of fermenting garlic, since garlic has toxicity as well as an antimicrobial property. Therefore, few studies on fermentation products of garlic have been made. The present inventors observed, however, that such problems could be overcome by lactic acid bacteria that is capable of fermenting garlic in Kimchi. The present inventors found that an excellent antiviral agent can be produced by fermenting garlic with lactic acid bacteria in Kimchi, to complete the present invention. It is thus expected that antiviral substances according to the present invention can replace currently used antiviral reagents that involve resistance problems.

An embodiment of the present invention provides a natural antiviral agent including one or more selected from the group consisting of a fermentation product of garlic fermented by using lactic acid bacteria, a concentrate of the same, a dried substance of the same, and a combination thereof, as an active ingredient.

Another embodiment of the present invention provides a composition for preventing and/or treating viral infection, which includes one or more selected from the group consisting of a fermentation product of garlic fermented by using lactic acid bacteria, a concentrate of the same, a dried substance of the same, and a combination thereof.

Another embodiment of the present invention provides a method of preventing and/or treating viral infection by using a natural antiviral substance including one or more selected from the group consisting of a fermentation product of garlic fermented by using lactic acid bacteria, a concentrate of the same, a dried substance of the same, and a combination thereof.

A more detailed description of the present invention is provided below. For the purpose of the present invention, lactic acid bacteria to be used may be one or more selected from the group consisting of bacteria belonging to genera Weissella, Lactococcus, and Leuconostoc. Lactic acid bacteria belonging to genus Weissella may include, but are not limited to, Weissella koreensis, Weissella cibaria, Weissella confusa, and like. For example, Weissella koreensis (KCTC 3621) was used as a representative strain of the genus of Weissella in a concrete embodiment of the present invention.

Lactic acid bacteria belonging to said genus of Lactococcus may include, but are not limited to, Lactococcus lactis spp. lactis, Lactococcus lactis spp. cremoris, and the like. For example, Lactococcus lactis spp. lactis (KCTC 3769) was used as a representative strain of the genus of Lactococcus in a concrete embodiment of the present invention.

Lactic acid bacteria belonging to said genus of Leuconostoc may include, but are not limited to, Leuconostoc lactis, Leuconostoc citreum, Leuconostoc mesenteroides, and like. For example, Leuconostoc lactis (KCTC 3528) was used as a representative strain of the genus of Leuconostoc in a concrete embodiment of the present invention.

Viruses against which the natural antiviral agents according to the embodiments of the present invention have antiviral activities include all types of pathogenic viruses, for example one or more selected from the group consisting of influenza viruses.

Said influenza viruses are influenza viruses that infect crustaceans or vertebrate animals such as fish, birds and mammals, and preferably shrimp, eels, chickens, ducks, pheasants, swine, bovines, canines, or humans, including influenza virus type A (Flu A), influenza virus type B (Flu B), and the like. Influenza virus type A includes combinations of 16 types of HA and 9 types of NA (144 subtypes), and avian influenza virus belongs to this type A. Among the viruses, types H1N1, H3N2, and B (Panama) were employed as representative viruses in a concrete embodiment, but the viruses to which the antiviral agent can be applied are not limited thereto.

For an embodiment of the present invention, the fermentation product of garlic may be obtained by fermenting garlic with lactic acid bacteria, in a cell-containing and/or cell-free form. The concentrate or dried substance of the fermentation product of garlic may be obtained through conventional methods for concentrations or drying as known in the art.

For an embodiment of the present invention, the fermentation product of garlic may be the fermentation product itself obtained by fermentation with the lactic acid bacteria, or its supernatant collected after centrifuging the fermentation product. In order to make a proper pH condition and obtain efficient antiviral activity, the fermentation product may be produced by adding water to ground garlic, and then fermenting them with said lactic acid bacteria, wherein the mixing ratio (by weight) of ground garlic and water may be 1:0.5 to 1.2 and preferably 1:0.8 to 1.2 (weight of ground garlic:weight of water), the amount of the lactic acid bacteria used may be 1.0×10⁶ cfu/g to 1.0×10¹² cfu/g (cfu per weight of mixture of ground garlic and water) and more preferably 1.0×10⁸ cfu/g to 1.0×10¹⁰ cfu/g, and the conditions may be a temperature of 20° C. to 40° C., more preferably 25° C. to 37° C., and a reaction time of 10 to 48 hours, and more preferably 18 to 30 hours.

The content of the fermentation product of garlic as an active ingredient in the antiviral agent according to the present invention may be at least 0.001 vol. %, preferably 1 vol. % or more, and more preferably 3 vol. % or more. The higher the content of the active ingredient is, the greater its antiviral effect will be, and the content to be applied may be properly adjusted depending on purposes, application forms, and desired effects of the antiviral agent, within the range below to the maximum of 100 vol. %. In an embodiment of the present invention, it was observed that antiviral activity was fully (100%) achieved at the content of about 5 vol. % or more, and thus the maximum concentration of the active ingredient in an antiviral agent may be any value within the range of 5 to 100 vol. %, and more preferably 7 to 100 vol. %. For example, content of the active ingredient in the antiviral agent may be within the ranges of about 0.1 to 30 vol. %, 1 to 30 vol. %, 1 to 20 vol. %, 3 to 30 vol. %, 3 to 20 vol. %, 3 to 10 vol. %, 5 to 30 vol. %, 5 to 20 vol. %, 5 to 10 vol. %, 7 to 30 vol. %, 7 to 20 vol. %, or 7 to 10 vol. %.

The dried substance that is obtained by drying the fermentation product of garlic fermented by lactic acid bacteria is obtained with the proportion of about 100 mg per 1 ml of said fermentation product of garlic. Therefore, the lower limit of the content of the fermentation product of garlic, the concentrate of the same, or the dried substance of the same may be at least 0.0001 wt. %, preferably at least 0.1 wt. %, and more preferably at least 0.3 wt. %, and the upper limit may be any value within 0.5 to 100 wt. %, and preferably 0.7 to 100 wt. %, based on dry weight. For example, the content of the active ingredient may be about 0.01 to 3 wt. %, 0.1 to 3 wt. %, 0.1 to 2 wt. %, 0.3 to 3 wt. %, 0.3 to 2 wt. %, 0.3 to 1 wt. %, 0.5 to 3 wt. %, 0.5 to 2 wt. %, 0.5 to 1 wt. %, 0.7 to 3 wt. %, 0.7 to 2 wt. %, or 0.7 to 1 wt. %.

The antiviral composition according to the present invention may be provided in a form selected from the group consisting of solution, feed, food, medication, and cosmetics, depending on the subject to which application will be made and the form of application. The content of the fermentation product of garlic fermented by lactic acid bacteria, the concentrate of the same, and/or the dried substance of the same in the antiviral composition may be properly adjusted depending on the application form of the composition. The contents may be within, but are not limited to, the range of 0.01 to 99.9 vol. % by dry volume, preferably 0.1 to 50 wt. %. The antiviral composition may further include additives such as excipients, preservatives, and stabilizers in conventional amounts, depending on the application form of the composition.

Another aspect of the present invention relates to a method for preventing and/or treating viral infection, which may include the step of applying the above antiviral agent and/or composition to an animal. Animals to which application is made may be any animal, preferably vertebrates such as crustaceans, fish, birds, and mammals, and more preferably shrimp, eels, chickens, ducks, pheasants, swine, bovines, canines, or humans. The application route may be any conventional administration route including all oral routes and all parenteral routes such as rectal, intravenous, intramuscular, hypodermic, and the like. Daily dose can be adjusted according to subject's symptoms, age, and condition, and desired effect from the application. For example, by weight of the active ingredient, a daily dosage can be within the range of 0.1 to 500 mg/kg (weight) and preferably 1 to 100 mg/kg (weight), which can be administered at a single dose or multiple doses, although dose amounts and administration regimes are not limited to the example.

As mentioned above, the fermentation product of garlic according to the present invention not only exhibits a great antiviral activity against viruses, and more specifically influenza viruses, but also possesses lesser toxicity to animal cells than non-fermented garlic does. As such, the fermentation product of garlic according to the present invention can be used as a natural antiviral substance against viruses.

### EXAMPLES

The present invention is further explained in more detail with reference to the following examples. These examples, however, should not be interpreted as limiting the scope of the present invention in any manner.

### Example 1

### Culture of Lactic Acid Bacteria

Genuses Weissella, Lactobacillus, Lactococcus, Enterococcus, Streptococcus, and Leuconostoc were used to culture lactic acid bacteria. Genus Weissella includes Weissella koreensis, Weissella cibaria, Weissella confuse, and the like, wherein Weissella koreensis (KCTC 3621) was used as a representative example. Genus Lactococcus includes Lactococcus lactis spp. lactis, Lactococcus lactis spp. cremoris, and the like, wherein Lactococcus lactis spp. lactis (KCTC 3769) was used as a representative example. Genus Leuconostoc includes Leuconostoc lactis, Leuconostoc citreum, Leuconostoc mesenteroides, and the like, wherein Leuconostoc lactis (KCTC 3528) was used as a representative example.

2 L of MRS broth (Difco) was subjected to high pressure sterilization at 121° C. for 15 minutes, and then allotted at the amount of 200 mL. Each of Weissella koreensis (KCTC 3621), Lactococcus lactis spp. lactis (KCTC 3769), and Leuconostoc lactis (KCTC 3528) was inoculated in the allotted broth at the concentration of 10⁴ to 10⁶ cfu/ml, and subjected to shaking culture at 25° C. for 24 hours.

### Example 2

### Preparation of Fermented Solution of Garlic

Garlic (Allium sativum) and lactic acid bacteria, such as Weissella koreensis (KCTC 3621), Lactococcus lactis spp. lactis (KCTC 3769), and Leuconostoc lactis (KCTC 3528) were used in preparing fermented solutions of garlic.

1000 g of ground garlic was added to 1000 g of water, and then allotted in amounts of 200 g. 20 g of each of Weissella koreensis (KCTC 3621), Lactobacillus fermentum (KCTC 3112), Lactococcus lactis spp. lactis (KCTC 3769), and Leuconostoc lactis (KCTC 3528) was added to the allotted ground garlic solutions to ferment for 24 hours. Each obtained fermented solution was centrifuged at 10,000 rpm for 8 minutes, and the supernatant was collected and filtrated with a 0.2 μm polypropylene syringe filter, to be used as a fermented solution of garlic in the following examples.

### Example 3

### Antiviral Effects of Fermented Solution of Garlic Fermented by Lactic Acid Bacteria

The investigation of the antiviral effect of the fermented solution of garlic that was fermented by lactic acid bacteria was made against influenza viruses Type A and Type B. In this example, influenza viruses Type H1N1 (A/Taiwan/1/86), Type H3N2 (A/Seoul/11/88), and Type B (B/Panama/45/90) were employed as representative types of the influenza viruses.

The antiviral effect of the fermented solution of garlic fermented by lactic acid bacteria was determined by a CPE inhibition assay.

Viruses diluted with MEM were inoculated onto MDCK (Madin-Darby canine kidney) cells that stop growing in 96-well plates in the amount of 100 μL per well so that the concentration in each well was 100 CCID₅₀ (50% cell culture inhibitory dose), and then absorbed in a CO₂ incubator at 37° C. for 60 minutes. The virus solution was removed, and 100 μL of the fermented solution of garlic fermented by lactic acid bacteria or control drugs were added thereto in duplicate. After culturing for three days, the efficiencies were determined. As the control dugs, lactic acid (Fluka), which is a main product of ground garlic solution and lactic acid bacteria culture and has been known to have a sterilization effect, and ribavirin, amantadine, and oseltamivir, which are conventional anti-influenza agents, were used.

### Example 4

### Determination of Antiviral Effect and Cytotoxicity of the Fermented Solution of Garlic Fermented by Lactic Acid Bacteria

EC₅₀ (50% effective concentration) was determined by the concentration of the fermented solution of garlic making 50% of cells survive when viruses were inoculated and cultured according to Example 3, and then the fermented solution of garlic fermented by lactic acid bacteria was added thereto. In order to examine the effect of cytotoxicity of the fermented solution of garlic on the efficacy test result, cytotoxicity was also determined from mock-infected wells prepared by adding 100 μL of the fermented solution of garlic fermented by lactic acid bacteria or a control drug to MDCK (Madin-Darby canine kidney) cells that stop growing in duplicate without inoculating the virus and incubating for three days. CC₅₀ (50% cytotoxic concentration) was determined by the concentration of the fermented solution or drug making 50% of cells die by comparing the number of surviving cells in the mock-infected well treated with the fermented solution or drug to that in a non-treated control well.

That is, the antiviral activity was determined as follows:

$${EC}_{50} = \frac{A ( Drug / Virus ) - A ( Virus control )}{A ( Cell control ) - A ( Virus control )} \times 100 \%$$

(A: Absorbance of surviving cells)

wherein 100% of survival indicates 100% of antiviral activity, and 0% of survival indicates 0% of antiviral activity.

In addition, survival percent in the monk-infected well for determining cytotoxicity was determined as follows:

$${CC}_{50} = \frac{A ( Drug ) - A ( Blank )}{A ( Cell control ) - A ( Blank )} \times 100 \%$$

(A: Absorbance of surviving cells)

wherein 100% of survival indicates no cytotoxicity, and 0% of survival indicates the strongest cytotoxicity.

Selectivity index (SI, CC₅₀/EC₅₀) was determined from EC₅₀ determined from the antiviral effect and CC₅₀ determined from the cytotoxicity.

### Example 5

### Anti-Influenzas Activity of the Fermented Solution of Garlic Fermented by Lactic Acid Bacteria

Anti-influenza activity of the fermented solution of garlic fermented by lactic acid bacteria was determined according to Examples 3 and 4, and the obtained results are shown in Table 1.

TABLE 1

| | | | Antiviral activity (EC₅₀) | | | Selectivity index (SI) | | |
|---|---|---|---|---|---|---|---|---|
| | | Toxicity | Taiwan | Seoul | Panama | Taiwan | Seoul | Panama |
| No | Code | CC₅₀ | A/H1N1 | A/H3N2 | B | A/H1N1 | A/H3N2 | B |
| 1 | LSN-G00 | 0.962 | 0.102 | 0.111 | 0.283 | 9.4 | 8.7 | 3.4 |
| 2 | LSN-K01 | 13.107 | 0.081 | 0.049 | 0.333 | 161.8 | 267.5 | 39.4 |
| 3 | LSN-K02 | 10.642 | 1.304 | 0.994 | 6.838 | 13.0 | 14.1 | 2.1 |
| 4 | LSN-K03 | 7.472 | 0.242 | 0.444 | 1.266 | 31.7 | 21.7 | 6.2 |
| 5 | Lactic acid | 0.014 | >0.014 | >0.014 | >0.014 | <1.00 | <1.00 | <1.00 |
| 6 | Ribavirin | >100.00 | 6.54 | 2.88 | 2.29 | >15.3 | >34.7 | >43.7 |
| 7 | Amantadine | >100.00 | >100 | 0.49 | >100 | ND | >204.1 | ND |
| 8 | Oseltamivir | >100.00 | 16.35 | 0.49 | 10.14 | >6.1 | >202.4 | >9.9 |

In Table 1, LSN-G00 represents ground garlic solution, LSN-K01 represents the fermented solution of garlic fermented by Weissella koreensis (KCTC 3621), LSN-K02 represents the fermented solution of garlic fermented by Lactococcus lactis spp. lactis (KCTC 3769), LSN-K03 represents the fermented solution of garlic fermented by Leuconostoc lactis (KCTC 3528), and the values were averages of the experimental results obtained from 5 solutions allotted from each of the fermented solutions of garlic.

As shown in Table 1, the fermented solution of garlic fermented by lactic acid bacteria has equal antiviral activity (EC₅₀) to that of the ground garlic solution, while it has considerably decreased cytotoxicity (CC₅₀) compared with the ground garlic solution. Based on the above result, efficacy as a treatment (selectivity index) of the fermented solution of garlic fermented by lactic acid bacteria is increased 2-17 times compared with that of the ground garlic solution. In the case of lactic acid that is a main product of lactic acid bacteria, since cytotoxicity is considerably strong (CC₅₀=0.014), antiviral activity (EC₅₀) cannot be calculated. The fermented solution of garlic fermented by lactic acid bacteria exhibits antiviral activity and a high selectivity index indicating efficacy as a antiviral agent, even at a small administration amount of 0.081 to 1.304 wt % compared with the conventional anti-influenza agent (0.49 to 100 wt %). In conclusion, the fermented solution of garlic fermented by lactic acid bacteria has excellent antiviral activity against influenza virus.

What is claimed is:

1. A method for preventing or treating viral infection, which comprises a step of applying an antiviral agent comprising one or more selected from the group consisting of a fermentation product of garlic fermented by lactic acid bacteria, and a concentrate and a dried substance thereof, as an active ingredient, to an animal.

2. The method according to claim 1, wherein said lactic acid bacteria is one or more selected from the group consisting of bacteria belonging to genus Weissella, Lactococcus, and Leuconostoc.

3. The method according to claim 2, wherein:
said lactic acid bacteria belonging to the genus Weissella is one or more selected from the group consisting of Weissella koreensis, Weissella cibaria, and Weissella confuse;
said lactic acid bacteria belonging to the genus Lactococcus is one or more selected from the group consisting of Lactococcus lactis spp. lactis, and Lactococcus lactis spp. Cremoris; and
said lactic acid bacteria belonging to the genus Leuconostoc is one or more selected from the group consisting of Leuconostoc lactis, Leuconostoc citreum, and Leuconostoc mesenteroides.

4. The method according to claim 3, wherein said lactic acid bacteria is one or more selected from the group consisting of Weissella koreensis (KCTC 3621), Lactococcus lactis spp. lactis (KCTC 3769), and Leuconostoc lactis (KCTC 3528).

5. The method according to claim 1, wherein said viral infection is caused by one or more viruses selected from the group consisting of influenza viruses Type A and influenza viruses Type B.

6. The method according to claim 1, wherein said fermentation product of garlic is obtained by fermenting garlic with lactic acid bacteria, in a cell-containing or cell-free form.
